(19) United States
(12) Patent Application Publication
(10) Pub. No.: US 2016/0022310 A1
(43) Pub. Date: Jan. 28, 2016

(54) TROCAR

(71) Applicants: DEMCON ADVANCED MECHATRONICS B.V., Enschede (NL); ACADEMISCH ZIEKENHUIS GRONINGEN, Groningen (NL)

(72) Inventors: Jonathan Johannes Zandman, Hardenberg (NL); Wouter Hagedoorn, Deventer (NL); Theodorus Johannes Klinkenberg, Haren (NL); Massimo Mariani, Haren (NL); Michiel Johannes Antonius Jannink, Almelo (NL)

(21) Appl. No.: 14/782,256

(22) PCT Filed: Apr. 4, 2014
(86) PCT No.: PCT/EP2014/056797

§ 371 Date: Oct. 2, 2015

(30) Foreign Application Priority Data
Apr. 4, 2013 (EP) 13162298.7

(51) Int. Cl. A61B 17/34

(52) U.S. Cl. CPC A61B 17/3421; A61B 2017/3427; A61B 2017/3445; A61B 2017/3466; A61M 13/00

(57) ABSTRACT

Trocar for instrument access to body cavities in minimally invasive surgery, the trocar comprising:—a body having a first access opening and at least one second access opening, each for inserting an instrument;—a cannula arranged with a proximal end to the body and having a distal end suitable for insertion into body cavities in minimally invasive surgery, wherein the cannula has a substantially constant out of round cross-section along is length, wherein a channel in the cannula is connected to the first access opening to provide a first passageway for an instrument and to the second access opening to provide at least one second passageway separate from said first passageway; and—a gas inlet arranged at the body and in fluid connection with the at least one passageway for inflating the body cavity with a gas, such as carbon dioxide gas.

The invention relates to a trocar for instrument access to body cavities in surgery, the trocar comprising:
a body having a first access opening for inserting an instrument; and
a cannula arranged with a proximal end to the body and having a distal end suitable for insertion into body cavities in surgery, wherein the channel in the cannula is connected to the first access opening to provide a passageway for the instrument.

Trocars are used to insert instruments into body cavities, like the abdomen or the chest, in minimally invasive surgery, for example, endoscopic surgery. In order to have sufficient space to operate, the body cavity is typically inflated with an inflation gas. This gas is fed through the cannula into the body cavity.

During for example endoscopic surgery, at least a camera and a tool are inserted through trocars into the body cavity. The size of the trocar and in particular of the canulla is selected based on the size of the tools to be inserted. Because all known trocars have a cylindrical canulla, the size of the trocar is selected based on the diameter of the canulla.

When performing endoscopic surgery, it is not always possible to simply select a larger trocar to fit all the instruments needed during surgery. During surgery in the chest, a trocar is inserted in between the ribs of the patient. The spacing of the ribs determines the maximum diameter of the canulla of the trocar. If the instruments to be used, do not fit through a single trocar and a larger trocar cannot be used due to the restriction by the spacing of the ribs, a second or a third trocar is used, which is also inserted in between the ribs. Insertion of a second or a third trocar will need additional incisions in the skin of the patient and will result in additional scar and pain.

When all instruments, including the camera are inserted through a single trocar, the instruments will all arrive at the same location inside the body. However, instruments entering the body through a second trocar, will arrive at a slightly different location and at a different angle. In order to have the instruments of the second trocar at the same spot as the instruments from the first trocar, additional effort is needed and the camera is needed to ensure that the instruments of the second trocar do not damage something when navigating the instruments.

It is an object of the invention to provide a trocar in which the above mentioned disadvantages are reduced or even removed.

This object, amongst other objects, is achieved according to the invention with a trocar according to appended claim 1. More specifically, this object, amongst other objects, is achieved by a trocar for instrument access to body cavities in minimally invasive surgery, the trocar comprising:
a body having a first access opening and at least one second access opening, each for inserting an instrument;
a cannula arranged with a proximal end to the body and having a distal end suitable for insertion into body cavities in minimally invasive surgery, wherein the cannula has a substantially constant out of round cross-section along its length, wherein a channel in the cannula is connected to the first access opening to provide a first passageway for an instrument and to the second access opening to provide at least one second passageway separate from said first passageway; and
a gas inlet arranged at the body and in fluid connection with the at least one passageway for inflating the body cavity with a gas, such as carbon dioxide gas.

The circular cross-section of the known prior art trocars limits the size of the passageway, when the diameter is restricted by for example the spacing of ribs. According to the invention, the cannula is provided with an out of round cross-section. So, if the height of the cross section is limited, for example by the spacing of the ribs, the width can be extended to provide for the required space within the cannula.

An added advantage of an out of round cross-section lies the way an incision behaves. Normally an incision is made along Langer's or Kraissl's lines to minimize damage from the incision itself. These lines lie along the orientation of collagen fibers in the dermis and epidermis. Because of this non-uniformity in the skin, the incision will open up in an ellipse-like shape. Conforming to this ellipse-like shape, an out of round cross-section will minimize wound-stress and thus reduce pain after the operation. To further minimize damage to the tissue, the cannula preferably has a blunt distal end.

The cannula has a substantially constant out of round cross-section along at least a part of its length, preferably along is entire length. The shape and size of the cross-section, in particular the outer contour thereof, is hereby the same along the length, such that the insertion depth of the cannula is not limited by its outer contour. Moreover, the size of the incision, though which the cannula extends into the body, is hereby substantially the same for each insertion depth. It is however noted that the distal end of the cannula may comprise an oblique surface, i.e. a surface under an angle with respect to the longitudinal axis of the cannula for easy insertion of the cannula in the patient.

The cross-section of the cannula has preferably a main axis and a minor axis perpendicular to the main axis and wherein the main dimension of the cross-section is different from the minor dimension of the cross-section. Preferably, the main dimension is at least two times the minor dimension. This embodiment provides at least twice the space for instruments than a trocar according to the prior art with a circular cross-section. The out of round cross-section of the cannula could have any shape except for a round shape. Preferred shapes for the cross section are an ellipse-shaped or rectangular-shaped cross section.

By providing two separate passageways, it is ensured, that two instruments simultaneously being arranged in the channel of the cannula, do not get in contact with each other and ensure that no conflict between the instruments arises when performing surgery. As the trocar is specifically intended for accessing body cavities in minimally invasive surgery, more preferably for thoracic surgery, each of the passageways is according to a preferred embodiment shaped and arranged to receive an instrument for performing minimally invasive surgery. The passageways thereto at least circumscribe an inner diameter, or preferably have an inner diameter, of at least 2.8 mm, a typical minimal outer diameter of instruments used in minimally invasive surgery. It is further noted that the term instrument also encompasses a camera which are typically used during minimally invasive surgery. The access openings and the associated passageways, or at least one thereof, are therefor preferably arranged for inserting and receiving a camera for use in minimally invasive surgery.

The separate passageways may be formed as tubular members for instance as integral parts of the cannula. The passageways may have substantially circular cross sections. It is also possible to form the passageways by separate tubular members arranged in the channel of the cannula for forming the two separate passageways. The tubular members may for instance be glued or otherwise arranged in said cannula. Yet another embodiment of the trocar according to the invention however comprises at least one longitudinal divider arranged in the channel of the canulla to provide at the least two separate passageways. By providing a divider, for instance in the form of a wall, two passageways are efficiently provided and it is ensured, that two instruments simultaneously being arranged in the channel of the canulla, do not get in contact with each other and ensure, that no conflict between the instruments arises when performing surgery.

According to a preferred embodiment, the first and second passageways extend parallel to each other along at least a length of the distal part of the cannula. The angle of the instruments arriving at the tissue is hereby the same. The working depth of the instruments in the body is furthermore not limited by the construction of the cannula, as the instruments may be extended from distal end of the cannula without unintentional interference. The passageways hereto preferably extend parallel to each other along the entire length of the cannula, i.e. from the body to the distal end thereof.

According to the invention, the body comprises a second access opening connected to one of the passageways. By providing two separate access openings, two instruments can be fed into the trocar to their respective passageways without difficulty. Preferably, the first and second access openings are at an angle with each other. This ensures that the proximal ends of the instruments, which often comprise a housing with control means, can be kept at a distance from each other. The openings communicate with the respective passageways, which preferably extend substantially parallel to each other as mentioned above. The channels defined by the openings and the passageways then preferably run parallel to each other in at least the distal parts of the cannula, more preferably along its entire length, to diverge from each other due to the openings provided at an angle with respect to each other at the proximal side of the trocar.

In yet another embodiment of the trocar according to the invention, sealing means are arranged in the at least one passageway. These sealing means ensure that the gas used to inflate the body cavity does not escape through the cannula and the access opening.

By providing a gas inlet arranged at the body and in fluid connection with the at least one passageway for inflating the body cavity with a gas, such as carbon dioxide gas, the trocar according to the invention can efficiently be used during minimally invasive surgery, as the gas supplied under pressure, and preferably sealed inside the body cavity using the sealing means as described above, allows inflating the body cavity for easy access of the instruments and for increasing the work space. A compact composition is obtained if the gas inlet is in fluid connection with and ends in a proximal part of the passageway. A passage for the gas hereby extends from an outer surface of the body through said body and opens out in a proximal part of the passageway. An additional passage for gas running parallel to the at least first and second passageways in the cannula is then not necessary.

These and other features of the invention will be elucidated in conjunction with the accompanying drawings.

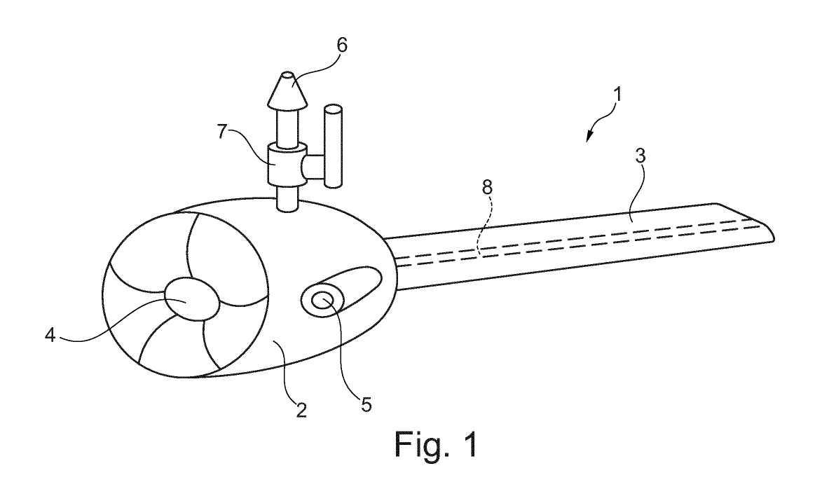

FIG. 1 shows an embodiment of a trocar according to the invention.

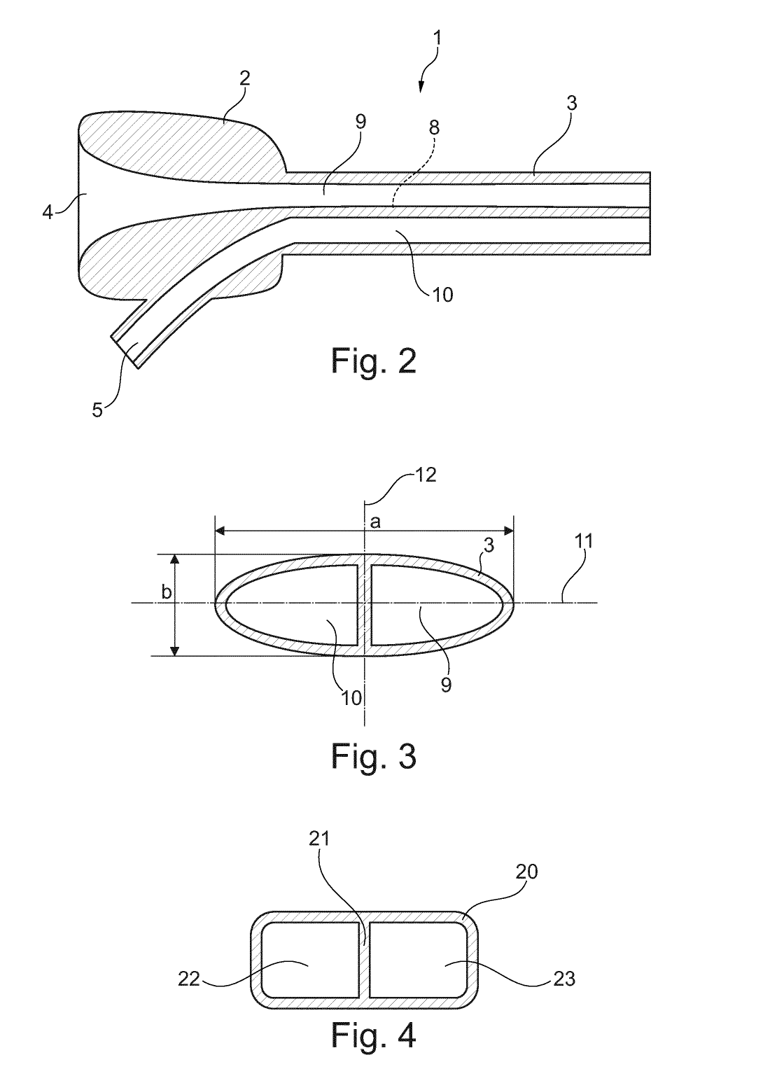

FIG. 2 shows a top cross sectional view of the trocar of FIG. 1.

FIG. 3 shows a cross sectional view of the cannula of the trocar of FIG. 1.

FIG. 4 shows a cross sectional view of a cannula of a second embodiment of the trocar according to the invention.

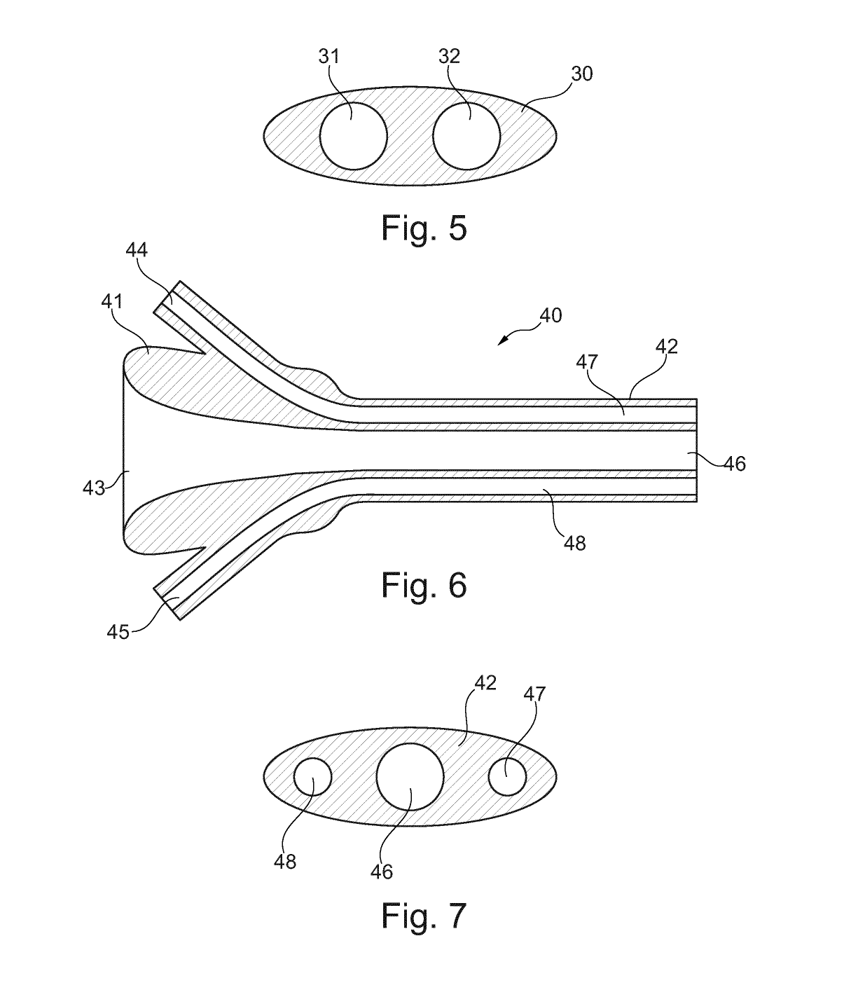

FIG. 5 shows a cross sectional view of a cannula of a third embodiment of the trocar according to the invention.

FIG. 6 shows a top cross sectional view of a fourth embodiment of the trocar according to the invention.

FIG. 7 shows the cross sectional view of the cannula of the trocar according to FIG. 6.

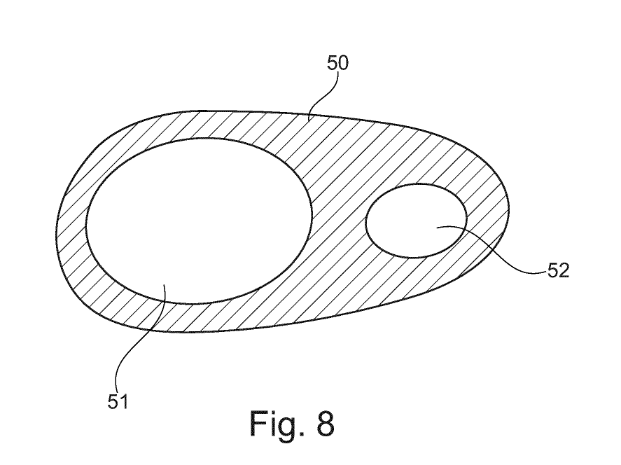

FIG. 8 shows a cross sectional view of a fifth embodiment of the trocar according to the invention.

FIG. 1 shows a perspective view of a first embodiment of a trocar 1 according to the invention. The trocar 1 has a body 2 with a cannula 3 arranged thereto. The body 2 has a first access opening 4 and a second access opening 5 for insertion of tools into the trocar 1. Furthermore, an inflation gas inlet 6 is arranged at the body 2. This inlet 6 is provided with a valve 7 to open or close the inlet 6.

As shown in FIG. 2, the cannula 3 is provided with a longitudinal divider wall 8, which provides two passageways 9, 10 within the cannula 3. The first passageway 9 is connected to the first access opening 4, while the second passageway 10 is connected to the second access opening 5. By providing two separate passageways 9, 10 it is ensured, that instruments inserted into the passageways 9, 10 cannot get in to contact with each other. As is clear from FIG. 2, the second access opening 5 is arranged at an angle with the longitudinal axis of the trocar 1. As a result, modules which are arranged on the proximal ends of the instruments, like a camera module or control module, are kept at a distance and do not interfere when surgery is performed. FIG. 3 shows a cross sectional view of the cannula 3. The cross section has a main axis 11 with a main dimension a and a minor axis 12 with a minor dimension b. In this embodiment the main dimension a is larger than twice the minor dimension b.

When the trocar 1 according to the invention is to be inserted between ribs, the ribs will limit the maximum height b of the cannula 3. However, the width a can still be chosen freely to meet the requirement of the number of instruments to be inserted.

FIG. 4 shows a cross sectional view of a cannula 20 of a second embodiment of the trocar according to the invention. This cannula 20 has a rectangular cross section. A divider wall 21 is arranged inside the channel of the cannula 20, to provide two separate passageways 22, 23.

FIG. 5 shows a cross sectional view of a cannula 30 of a third embodiment of the trocar according to the invention. The cannula 30 has two separate passageways 31, 32. The passageways 31, 32 each have a circular cross section, which ensures that the tools running through the passageways 31, 32 are not influenced by the shape of the cross section of the passageways 31, 32. An out of round cross-section of a passageway could have an impact on for example rotating a tool.

FIG. 6 shows a top cross sectional view of a fourth embodiment of the trocar 40 according to the invention. This trocar 40 a body 41 with a cannula 42 arranged thereto. The body 41 has a first access opening 43, a second access opening 44 and a third access opening 45 for insertion of tools into the trocar 1. The access openings 43, 44, 45 are connected to respective passageways 46, 47, 48 in the cannula 42. The two outer passageways 47, 48 have in this embodiment a smaller cross section than the central passageway 46 (see also FIG. 7).

FIG. 8 shows a cross sectional view of the cannula 50 of a fifth embodiment of the trocar according to the invention. The cross section of the cannula 50 is oval and comprises a main passageway 51 and a sub passageway 52.

The present invention is not limited to the embodiment shown, but extends also to other embodiments falling within the scope of the appended claims. For instance, although the embodiments shown in the figures show a cannula having two or three separate passageways, it is also within the scope of the invention to provide a single passageway or to provide four or more passageways.

1. Trocar for instrument access to body cavities in minimally invasive surgery, the trocar comprising:
a body having a first access opening and at least one second access opening, each for inserting an instrument;
a cannula arranged with a proximal end to the body and having a distal end suitable for insertion into body cavities in minimally invasive surgery, wherein the cannula has a substantially constant out of round cross-section along is length, wherein a channel in the cannula is connected to the first access opening to provide a first passageway for an instrument and to the second access opening to provide at least one second passageway separate from said first passageway; and
a gas inlet arranged at the body and in fluid connection with the at least one passageway for inflating the body cavity with a gas, such as carbon dioxide gas.

2. Trocar according to claim 1, wherein the cross-section of the cannula has a main axis and a minor axis perpendicular to the main axis and wherein the main dimension of the cross-section is different from the minor dimension of the cross-section.

3. Trocar according to claim 2, wherein the main dimension is at least two times the minor dimension.

4. Trocar according to claim 1, wherein the cannula has an ellipse-shaped or rectangular-shaped cross section.

5. Trocar according to claim 1, wherein the first and second passageways extend parallel to each other along at least a length of the distal part of the cannula.

6. Trocar according to claim 1, wherein the first and second access openings are at an angle with each other.

7. Trocar according to claim 1, comprising at least one longitudinal divider arranged in the channel of the cannula to provide the at least two separate passageways.

8. Trocar according to claim 1, wherein sealing means are arranged in the at least one passageway.

9. Trocar according to claim 1, wherein the gas inlet is in fluid connection with and ends in a proximal part of the passageway.

10. Trocar according to claim 1, wherein the cannula has a blunt distal end.

11. Trocar according to claim 1, wherein the passageways have an inner diameter of at least approximately 2.8 mm.
